(19) United States
(12) Patent Application Publication
(10) Pub. No.: US 2010/0298764 A1
(43) Pub. Date: Nov. 25, 2010

(54) Fluid delivery system with optical sensing of analyte concentration levels

(76) Inventors: Ofer Yodfat, Modi'in (IL); Gavriel J. Iddan, Haifa (IL); Ruthy Kaidar, Haifa (IL); Gali Shapira, Haifa (IL)

Correspondence Address: MINTZ LEVIN COHN FERRIS GLOVSKY & POPEO, ONE FINANCIAL CENTER, BOSTON, MA, 02111, US

(21) Appl. No.: 11/989,678

(22) PCT Filed: Sep. 5, 2007
(86) PCT No.: PCT/IL2007/001096

§ 371 Date: Jul. 29, 2010

Related U.S. Application Data
(60) Provisional application No. 60/842,869, filed on Sep. 6, 2006.

(51) Int. Cl. A61M 5/168

(52) U.S. Cl. 604/66

(57) ABSTRACT

A system for continuous monitoring of body analytes and controlling delivery of fluids to a body of a user. The system includes a sensing apparatus configured to detect concentration level of analyte in the body of the user using optical means and a dispensing apparatus configured to infuse fluid into the body of the user based on the detected concentration level of analyte.

### FIELD OF INVENTION

The present invention generally relates to systems, methods, and apparatuses for continuous monitoring of body analytes and controlling delivery of fluids. In particular, the present invention relates to a closed loop system for monitoring glucose levels and controlling insulin delivery. Even more particularly, a spectroscopic-based continuous subcutaneous glucose monitoring system that can be coupled with an insulin delivery system.

### BACKGROUND OF THE INVENTION

### Diabetes and Glycemic Control

Diabetes mellitus is a disease of major global importance, increasing in frequency at almost epidemic rates, such that the worldwide prevalence in 2006 is 170 million people and predicted to at least double over the next 10-15 years. Diabetes is characterized by a chronically raised blood glucose concentration (hyperglycemia), due to a relative or absolute lack of the pancreatic hormone, insulin. Within the healthy pancreas, beta cells, located in the islets of Langerhans, continuously produce and secrete insulin according to the blood glucose levels, maintaining near constant glucose levels in the body.

Much of the burden of the disease to the patient and to health care resources is due to the long-term tissue complications, which affect both the small blood vessels (microangiopathy, causing eye, kidney and nerve damage) and the large blood vessels (causing accelerated atherosclerosis, with increased rates of coronary heart disease, peripheral vascular disease and stroke). There is now good evidence that morbidity and mortality of diabetic patients is related to the duration and severity of hyperglycemia (DCCT Trial, N. Engl. J. Med. 1993; 329: 977-986, UKPDS Trial, Lancet 1998; 352: 837-853, BMJ 1998; 317, (7160): 703-13 and the EDIC Trial, N. Engl. J. Med. 2005; 353, (25): 2643-53].

In theory, returning blood glucose levels to normal by hormone replacement therapy using insulin injections and/or other treatments in diabetes should prevent complications, but, frustratingly, near-normal blood glucose concentrations are very difficult to achieve and maintain in many patients, particularly those with type 1 diabetes. In these patients, blood glucose concentration can swing between very high (hyperglycemia) and very low (hypoglycemia) levels in an unpredictable manner. Thus, tight glycemic control is required. This control can be achieved by substituting the two functions of the normal pancreas—glucose monitoring and insulin delivery. Furthermore, a closed loop system provided with a feedback mechanism connecting between both functions (often referred to as an “artificial pancreas”) could theoretically maintain near normal blood glucose levels.

### Glucose Monitoring

Most diabetic patients currently measure their own blood glucose level discontinuously, i.e., several times during the day by obtaining finger-prick capillary samples and applying the blood to a reagent strip for analysis in a portable meter. Unfortunately the discomfort involved leads to poor patient compliance. Testing cannot be performed while sleeping and while the subject is occupied. In addition, the results do not give information regarding the trends in glucose levels, but rather provide only discrete readings, taken at large time intervals from one another. Therefore, continuous glucose monitoring is advantageous, providing essentially continuous glucose readings buy performing discrete measurements, at a very high rate. Continuous monitoring and can be done by invasive, minimally-invasive, or non-invasive means.

### Invasive Continuous Glucose Monitoring

Invasive continuous glucose monitoring involves the implantation of a sensing device in the body. As detailed in U.S. Pat. Nos. 6,122,536 to Sun and 6,049,727 to Crothall, both assigned to Animas Corporation, an invasive spectroscopy-based glucose sensor, designed for long-term (>5 years) internal use is under development. The Animas sensor has the advantage of being able to directly read glucose in the blood. A small, ultralight C-clamp detector is surgically implanted around a 4-5 mm (0.2 inch) diameter blood vessel. The detector has two tiny probes at the tips of the C-clamp structure which puncture each side of the vessel and allow transmission of a clean infrared light signal between them. A larger device housing a laser generator plus signal analysis is located nearby within a closed compartment under the skin. The laser IR signal is transmitted to the detector around the vessel and returns the transmitted beam back to the processing unit. Readings are available at short time intervals. Major advantages of this approach are that calibration is required only once a week and that although minor surgery is required, this sensor provides direct access to blood.

Minimally-invasive glucose monitoring

Minimally-invasive glucose monitors measure glucose levels in the interstitial fluid (ISF) within the subcutaneous tissue. The strong correlation between blood and ISF glucose levels, allows for accurate glucose measurements (Diabetologia 1992; 35, (12): 1177-1180).
GlucoWatch® G2 Biographer is one commercially available minimally-invasive glucose monitor. GlucoWatch is based on reverse iontophoresis as disclosed in U.S. Pat. No. 6,391,643, assigned to Cygnus Inc. A small current passed between two skin-surface electrodes draws ions and (by electro-endosmosis) glucose-containing interstitial fluid to the surface and into hydrogel pads provided with a glucose oxidase (GOX) biosensor (JAMA 1999; 282: 1839-1844). Readings are taken every 10 min, with a single capillary blood calibration. Disadvantages of the GlucoWatch include occasional sensor values differing markedly from blood values; skin rashes and irritation in those locations which are immediately underneath the device, appearing in many users; a long warm up time of 3 hours; and skips in measurements due to sweating.
Another commercially available minimally-invasive monitor is the Guardian® RT Continuous Glucose Monitoring System, developed by Medtronic MiniMed Inc. Guardian® RT is a GOX-based sensor, (as discussed in U.S. Pat. No. 6,892,085). The sensor consists of a subcutaneously implanted, needle-type, amperometric enzyme electrode, coupled with a portable logger (Diab Tech Ther 2000; 2: Supp. 1, 13-18). The Guardian® RT system displays updated glucose readings every five minutes, together with hypo- and hyperglycemic alarms. The sensor is based on the long-established technology of GOX immobilized at a positively charged base electrode, with electrochemical detection of hydrogen peroxide production.
The Freestyle Navigator™ is another GOX-based sensor, discussed in U.S. Pat. No. 6,881,551 to Heller, assigned to Abbott Laboratories, formerly TheraSense, Inc. This sensor is placed just under the skin by a disposable self-insertion device. Information is communicated wirelessly between the transmitter and the receiver every minute. The receiver is designed to display glucose values, directional glucose trend arrows, and rate of change. The receiver also has high and low glucose alarms, and stores glucose data for future analysis.
U.S. Pat. No. 6,862,465 to Shults and U.S. Patent Publication No. 2006/0036145 to Brister, assigned to DexCom, discuss long- and short-term GOX-based continuous glucose monitoring systems. Both systems include a sensor, a small insertable or implantable device that continuously measures glucose levels in subcutaneous tissue, and a small external receiver to which the sensor transmits glucose levels at specified intervals. The receiver displays the patient's current blood glucose value, as well as 1-hour, 3-hour and 9-hour trends. The receiver also sounds an alert when an inappropriately high or low glucose excursion is detected. The DexCom™ STS™ Continuous Glucose Monitoring System is a user insertable short-term sensor that is inserted just under the skin where it is held in place by an adhesive. Once inserted the user would wear the sensor for up to three days before being replaced. After three days, the user removes the sensor from the skin and discards it. A new sensor can then be used with the same receiver. The DexCom™ STS™ Continuous Glucose Monitoring System has been FDA-approved. The DexCom™ Long Term Sensor is implanted under the skin in the abdomen by a local anesthetic short procedure carried out by a physician. This sensor is designed to function for up to one year. At the end of its life, the sensor can be removed by a physician in a short procedure, and another sensor implanted.

The enzymatic reaction that occurs in the above mentioned electrochemical sensors, catalyzed by GOX, consumes oxygen and glucose to yield gluconic acid and hydrogen peroxide, leading to numerous disadvantages inherent to glucose monitoring, which employs such reactions, including:
GOX-based devices rely on the use of oxygen as the physiological electron acceptor, and thus, are subject to errors due to fluctuations in the oxygen tension and the stoichiometric limitation of oxygen in vivo.
The amperometric measurement of hydrogen peroxide requires application of a potential at which additional electroactive species exist, e.g., ascorbic and uric acids or acetaminophen. These and other oxidizable constituents of biological fluids can compromise the selectivity and hence the overall accuracy of the glucose concentration measurement.
Hydrogen peroxide is known for its toxic effects compromising the biocompatibility of the sensor.
Hydrogen peroxide deactivates the GOX molecules, limiting the time available for application of the sensor.
The size of the cannula, including the sensing mechanism deployed within it is relatively large, compromising the ease and comfort of the cannula insertion into the user's body.
Miniaturizing the sensing technology within the cannula, which requires high levels of enzyme loading, while keeping high measurement sensitivity, remains a challenge.

Microdialysis is an additional commercially available minimally-invasive technology (Diab Care 2002; 25: 347-352) for glucose monitoring as discussed in U.S. Pat. No. 6,091,976 to Pfeiffer, assigned to Roche Diagnostics, and the marketed device: Menarini Diagnostics, GlucoDay® S. A fine, semi-permeable hollow dialysis fiber is implanted in the subcutaneous tissue and perfused with isotonic fluid. Glucose diffuses across the semi-permeable fiber and is pumped outside the body via the microdialysis mechanism for measurement by a glucose oxidase-based electrochemical sensor. Initial reports (Diab Care 2002; 25: 347-352) show good agreement between sensor and blood glucose readings, and good stability with a one-point calibration over one day. Higher accuracies were found when using the microdialysis-based sensor, compared to the needle-type sensor (Diabetes Care 2005; 28, (12): 2871-6).

Disadvantages of the microdialysis-based glucose sensors stem primarily from the constant perfusion of solution through the microdialysis probe. This operational method requires the presence of a dedicated pump and reservoir, leading to large and bulky devices, and also necessitates high energy consumption. Furthermore, the relatively large size of the microdialysis catheter often causes a wound and subsequent local tissue reactions, following its insertion into the subcutaneous tissue. Finally, the microdialysis process generates long measurement lag times, due to the essential slow perfusion rates and long tubing.

### Non-Invasive Continuous Glucose Monitoring

Non-invasive continuous glucose monitoring includes the sensing of glucose in blood, ISF or other physiological fluids, primarily using optical means. U.S. Pat. No. 6,928,311 to Pawluczyk, assigned to NIR Diagnostics Inc, describes a non-invasive monitor that uses near infrared light. A beam of light in the Near-IR range is focused on the person's finger for about half a minute. By applying mathematical algorithms on the emerging light signal, the concentration of various blood analytes including glucose are determined and displayed to the user.

Continuous glucose monitoring systems are calibrated relative to known glucose values for maintaining accurate glucose measurements throughout their operation. Calibration is performed by adjusting the measured value to a known standard value. Commercially available continuous glucose monitors, are often calibrated against blood glucose measurements, tested with a blood glucose meter, which involves finger-pricking, requiring several calibrations throughout the period of the sensor use. The need for these frequent invasive calibrations contradicts the fundamental purpose of continuous sensors, intended to eliminate users' noncompliance with finger-prick blood glucose tests, by providing alternative means for glucose monitoring.

Continuous glucose monitoring based on optical methods employs various sensing methodologies for measuring glucose concentration levels. Optical sensing methods are quite prevalent among glucose sensors and include NIR, IR, Raman, Polarimetry, and Photoacoustic technology.

In Near-Infrared (NIR) spectroscopy, a selected band of NIR light is transmitted through the sample, and the analyte concentration is obtained by the analysis of the resultant spectral information. The NIR absorbance bands tend to be broad and overlap, and are highly influenced by temperature, pH, and other physical factors. Nevertheless, the NIR spectrum allows for large optical path lengths to be used due to relatively easy passage through water (the light absorbance is directly proportional to the path length according to the Beer-Lambert law).

The near-infrared spectrum spans a wide range from 700 to 2500 nm. Absorption features throughout this spectral range primarily correspond to overtones and combinations of molecular vibrations. The absorption properties of water play a critical role in the regions of the near-infrared spectrum available for noninvasive measurements. Strong water absorption bands centered at approximately 1333, 1923, 2778 nm (7500, 5200, and 3600 cm¹) create three transmission windows through aqueous solutions and living tissue. These spectral windows are termed the short-wavelength region (700-1370 nm, 14 286-7300 cm⁻¹), the first overtone region (1538-1818 nm, 6500-5500 cm⁻¹), and the combination region (2000-2500 nm, 5000-4000 cm⁻¹). Absorption features in the combination region correspond to first-order combination transitions associated with bending and stretching vibrations of C—H, N—H, and O—H functional groups. The first overtone region corresponds to the first-order overtone of C—H stretching vibrations, and the shortwavelength region includes numerous higher order combination and overtone transitions. For combination spectra, molar absorptivities are larger and bands are narrower compared to first overtone spectral features. Near-infrared absorption features become significantly weaker and broader as the order increases, thereby greatly reducing the analytical utility of the shortwavelength region in terms of molecular vibrational information. (Anal Chem 2005 (77), pp. 5429-5439).

A relative dip in the water absorbance spectrum opens a unique window in the 2000-2500 nm wavelength region, saddled between two large water absorbance peaks. This window allows pathlengths or penetration depths on the order of millimeters and contains specific glucose peaks at 2130, 2270 and 2340 nm. This region offers the most promising results for quantifiable glucose measurements using NIR spectroscopy (Biomed Photonics Handbook, 2003, p. 18-13).

The different spectral regions permit for several sample volumes and optical path lengths: larger samples are possible for spectra collected at shorter wavelengths and longer wavelengths are restricted to smaller samples. Optimal sample thickness for the combination, first overtone, and short wavelength range are 1, 5, and 10 mm respectively. However, when the collected spectra encompass multiple spectral regions, it is not possible to match the sample thickness with each spectral region (Anal. Chem. 2005, 77, 5429-5439). Comparison between transmittance and reflectance measurements in glucose using near infrared spectroscopy shows that transmittance is preferred for glucose monitoring (Journal of Biomedical Optics 11(1), pp. 014022-1-7, January/February 2006). FIG. 1A illustrates optical absorption spectra of glucose in the NIR region for aqueous glucose after water subtraction. (Journal of Biomedical Optics 5(1), 5-16 January 2000)

In mid-Infrared (mid-IR) spectroscopy, the wavelengths of glucose absorbance in the mid-IR spectrum range (2500-10000 nm) are used for the analysis of glucose concentration. Although the absorption bands tend to be sharp and specific, there is strong background absorption by water that severely limits the optical path length that may be used. FIG. 1B illustrates optical absorption spectra of glucose in the Mid-IR region for aqueous glucose after water subtraction. (Journal of Biomedical Optics 5(1), 5-16 January 2000)

In Raman spectroscopy, Raman spectra are observed when incident light is inelastically scattered producing Stokes and anti-Stokes shifts, where the latter is the more prevalent. Raman spectra are less influenced by water compared to NIR/IR and the peaks are spectrally narrow. In addition, Raman spectroscopy requires minimal sample preparation. However, the signal is weak and therefore requires a highly sensitive detection system (CCD array).

It is possible to detect glucose by monitoring the 3448 nm (2900 cm⁻¹) C—H stretch band or the C—O and C—C stretch Raman bands at 8333-11111 nm (900-1200 cm⁻¹), which represents a fingerprint for glucose (Clinical Chemistry 45:2 165-177, 1999). FIG. 1C illustrates Raman spectrum for aqueous glucose, after subtraction of the water background. (Journal of Biomedical Optics 5(1), 5-16 January 2000)

Polarimetry involves the optical rotation of the polarized light by the chiral centers of glucose, which is determined by the structure of the molecule, the concentration of the molecule, and the optical path length the light traverses through the sample. Each optically active substance has its own specific rotation, as defined by Biot's law. The measurement of the optical rotation requires a very sensitive polarimeter, due to the low glucose concentrations in the cell. For example, at a wavelength of 670 nm, glucose will rotate the linear polarization of a light beam approximately 0.4 millidegrees per 10 mg/dl for a 1-cm sample pathlength (Biomedical Photonics Handbook, 2003, p-18-14). In addition, the presence of other optically active molecules make the accurate detection of glucose concentration complicated.

Finally, photoacoustic (PA) spectroscopy involves light which is absorbed by glucose, leading to thermal expansion and to the generation of a detectable ultrasound pressure wave. In one study, solutions of different glucose concentrations were excited by NIR laser pulses at wavelengths that corresponded to NIR absorption of glucose in the 1000-1800 nm range. There was a linear relationship between PA signal and glucose concentrations in aqueous solutions. (Diabetes Technology & Therapeutics, Vol. 6, November 2004, O. S. Khalil). This method is particularly sensitive to changes in temperature.

Optical glucose measurement techniques are particularly attractive for several reasons: they utilize nonionizing radiation to interrogate the sample, they do not generally require consumable reagents, and they are fast. Also, a use of optical glucose monitoring methods is attractive because they are nondestructive and reagentless, thereby eliminating the risk of unsafe reactions and their byproducts.

Although optical approaches for glucose sensing are attractive, they are nevertheless often plagued by a lack of sensitivity and/or specificity since variations in optical measurements depend on variations of many factors in addition to glucose concentration. Isolating those changes which are due to glucose alone and using them to predict glucose concentration is a significant challenge in itself. (Journal of Biomedical Optics 5(1), 5-16 January 2000). Furthermore, non-invasive optical glucose monitors, which involve sensing of glucose levels through the skin, involve very low signal-to-noise ratio, scattering and interferences by bodily fluids and by the skin itself, causing noninvasive optical sensors to lack specificity and repeatability.

Since prior art optical methods are usually used in noninvasive applications, they do not produce accurate and specific results. Thus, there is a need for an immediate application of the optical methods directly to the ISF or to fluids comprising endogenous components of the ISF, thus, eliminating the attenuating effects of the skin.

### Closed-Loop Systems

Continuous glucose monitoring alone is not sufficient for balanced diabetes management. Tight glycemic control can be achieved by substituting both functions of the normal pancreas, glucose sensing and insulin delivery. A closed loop system provided with a feedback mechanism could theoretically maintain near normal blood glucose levels. Such a closed loop system, referred to as an “artificial pancreas”, includes an insulin pump and a continuous glucose sensor that works together to imitate the human pancreas. The continuous glucose sensor reports the measured glucose values to the insulin pump, which then supplies the appropriate dose of insulin and delivers it to the user's body. In a semi-closed loop system, user inputs are added as supplementary inputs to the system, in addition to the continuously measured glucose values measured by the sensor, and both inputs are used for calculating appropriate insulin dosage.

Today, artificial pancreatic systems contain a sensor and a pump which are two separate components, where both are relatively bulky and heavy devices that are separately affixed to the patient's belt or pockets. In addition, the two devices have two separate infusion sets with long tubing and two insertion sites. As a consequence, the time for the devices' insertion and disconnection increases as well as the probability for adverse events like infections, irritations, bleeding, etc.

Thus, there is a need for a device that monitors glucose levels and concomitantly delivers insulin, being a miniature single device, discreet, economical for the users and highly cost effective.

There is also a need for a closed loop system that monitors glucose levels and dispenses insulin according to the sensed glucose levels. In some embodiments, the system can be a miniature single device, discreet, economical for the users and highly cost effective for the payer.

There is also a need for a fluid delivery device that can concomitantly dispense insulin and monitor glucose at the same (insertion) site.

There is also a need for a method, which allows to dispense insulin and monitor glucose using a single subcutaneous cannula, avoiding multiple painful skin pricking.

There is also a need for an accurate, reliable, minimally-invasive, continuous glucose monitor, based on an optical measurement, avoiding any direct contact between the sensed fluid and the sensing means.

There is also a need for a glucose monitor that can be configured to provide immediate interaction between the light produced by an optical sensing means and the measured analyte.

There is also a need in a method for monitoring analyte concentration that includes optically sensing the analyte concentration within a subcutaneous cannula by optical means.

There is also a need in a method for monitoring ISF analyte that includes optically sensing a subcutaneous ISF analyte, within a fluid that is transported outside of the body.

### SUMMARY OF THE INVENTION

It is an object of some of the embodiments of the present invention to provide an improved closed loop system that enables continuous, real-time monitoring of the analyte concentration levels in the body of a user.

It is an object of some of the embodiments of the present invention to provide a device that concomitantly dispenses insulin and monitors glucose levels.

It is an object of some of the embodiments of the present invention to provide a miniature skin adhered device that dispenses insulin and monitors glucose levels.

It is an object of some of the embodiments of the present invention to provide a device that dispenses insulin and monitors glucose using a single subcutaneous cannula.

It is an object of some of the embodiments of the present invention to measure analyte concentration levels continuously by performing discrete measurements, at a high measurement rate.

It is an object of some of the embodiments of the present invention to detect analyte concentration levels in the body by using optical detection means.

It is an object of some of the embodiments of the present invention to detect analyte concentration levels in the body by using optical means, capable of directly monitoring a subcutaneous ISF fluid, located below the skin.

It is an object of some of the embodiments of the present invention to detect analyte concentration levels in the body by using optical means, capable of directly monitoring a subcutaneous ISF fluid, or fluids having endogenous components of the ISF, inside the dispensing cannula.

It is another object of some of the embodiments of the present invention to provide a system for detecting analyte concentration levels in the body, where the system includes optical means, for directly monitoring a subcutaneous ISF fluid, or fluids having endogenous components of the ISF, and a means to transport said fluid.

It is an object of some of the embodiments of the present invention to provide a device that includes a disposable part and a reusable part. The reusable part can be configured to include relatively expensive components and the disposable part can be configured to include relatively cheap components, thereby, providing a low cost product for the user and a profitable product for the manufacturer and payer.

It is an object of some of the embodiments of the present invention to provide a method for sensing one or more body analytes including a combination of at least one or more optical methods, one or more non-optical physical methods and one or more electro-chemical methods for sensing one or more body analytes.

It is an object of some of the embodiments of the present invention to provide an apparatus for sensing one or more body analytes having a combination of at least one or more optical sensing means, one or more non-optical physical sensing means and one or more electro-chemical sensing means.

Some embodiments of the present invention relate to a closed loop system that regulates body analyte concentrations by concomitantly monitoring analyte levels and dispensing a fluid, e.g., a drug that can adjust the analyte levels.

Some embodiments of the present invention relate to a skin adherable device capable of irradiating light through a bodily compartment, or through an endogenous substance, and detecting the returned light, thus allowing monitoring of analyte concentrations by spectroscopic means.

In one embodiment, the device includes a dispensing apparatus and a sensing apparatus. The dispensing apparatus infuses a fluid into the body of a user. The sensing apparatus detects one or more analyte concentration levels in the body.

In an alternate embodiment, the dispensing apparatus and the sensing apparatus may work in a closed loop system, where a processor-controller apparatus regulates the dispensing of a fluid according to the sensed analyte concentration.

In another alternate embodiment, the dispensing apparatus and the sensing apparatus may work in a semi-closed loop system, where a processor-controller apparatus regulates the dispensing of the fluid according to the sensed analyte concentration and according to external user inputs.

In yet another alternate embodiment, the device includes two remotely controlled units, one unit containing the dispensing apparatus and another unit containing the sensing apparatus. The loop is closed by transmittance of information from the sensing apparatus to the dispensing apparatus, which adjusts delivery of the fluid accordingly.

In yet another alternate embodiment, the device includes a single unit that contains only a sensing apparatus. Thus, the device is a continuous analyte (e.g., glucose) monitoring system.

In one embodiment, the device comprises two parts, a reusable part having all of the electronic elements and all of the driving elements and a disposable part having a fluid reservoir and the needle assembly. The monitored analyte can be glucose. The dispensed fluid can be insulin, to be used with diabetic patients.

In another embodiment, the device includes a dispensing apparatus and a non-invasive sensing apparatus, in which detection of analyte concentration levels are performed non-invasively. Measurement of analyte concentrations is carried out without direct contact between the sensing apparatus and the interstitial fluid.

In another embodiment, the device includes a minimally-invasive sensing apparatus, in which detection of the analyte concentration levels is performed in a minimally-invasive manner. The skin adhered patch serves as a sensing device and comprises a single cannula, which is inserted into the subcutaneous tissue and monitors the ISF analyte levels.

In yet another embodiment, the device includes a dispensing apparatus and a minimally-invasive sensing apparatus, in which detection of analyte concentration levels is performed in a minimally-invasive manner. The minimally-invasive sensing apparatus can use micropores made in the skin to extract ISF from the body, thus overcoming the skin's highly scattering properties and increasing the accuracy of the optical measurements. Such micropores are made by means of laser, reverse iontophoresis or any other methods known in the art. Alternatively, the minimally-invasive sensing apparatus can use a cannula, inserted into the subcutaneous tissue allowing contact with the ISF.

In one embodiment, the adherable device includes a fluid reservoir, a needle assembly, a pumping apparatus and an optical sensing apparatus. The reservoir contains fluid, such as isotonic fluid or medication (e.g., insulin). The flow of fluid from the reservoir is controlled by the pumping apparatus and a processor-controller apparatus. The needle assembly includes a cannula and a penetrating member. The penetrating member is used to insert the cannula into the body.

In an alternate embodiment, the cannula is configured as a semi-permeable membrane enabling diffusion, and thus, selectively allowing entry of analyte molecules (e.g., glucose) into the cannula. This space is occupied either by an isotonic dispensed fluid, or by medication (e.g., insulin). The diffusion process, occurring across the semi-permeable membrane, allows analyte molecules (e.g., glucose) to move according to the concentration gradient and rapidly achieve partial or full equilibrium, i.e., the analyte concentration in the fluid within the cannula, is proportional or equal to the analyte concentration in the interstitial fluid (ISF) outside the cannula.

The membrane constructing the cannula is permeable, enabling diffusion, and non-selective entry of analyte molecules (i.e., glucose molecules and other molecules contained in the ISF) into the cannula.

The sensing of glucose levels and the dispensing of insulin are both done through one single exit port, using a single cannula, in some embodiments of the invention. The sensing apparatus and dispensing apparatus share a cannula, a fluid reservoir, and a pump. Thus, the device contains a single cannula, a single fluid reservoir and a single pump.

In another embodiment, the device includes two exist ports. Monitoring analyte (e.g., glucose) levels is effected through a single exit port (a single cannula) and the dispensing of fluid (e.g., insulin) is carried out through another (exit) port, using an additional cannula. Accordingly, in such an embodiment, the sensing apparatus and the dispensing apparatus have separate cannulae and associated separate fluid reservoirs. Fluid delivery (pumping) from both reservoirs can be achieved either by one pump or by two separate pumps.

In some embodiments, the pumping mechanism is peristaltic. Both in the single cannula and in the double cannula configurations, a single peristaltic wheel can dispense fluid through one or more delivery tubes.

In still other embodiments, two peristaltic pumps may be used: one pump is used with a tube used for fluid delivery, and another pump is used with a tube for analyte levels sensing.

In yet another embodiment, a pump that contains a syringe reservoir may be used. In this case, two pumping mechanisms and two syringe reservoirs may be used for the double cannula configuration.

According to some of the embodiments of the present invention, the sensing apparatus is based on optical detecting methods, using the optical properties of the monitored analyte (e.g., glucose). The optical detecting method is based on at least one method from the group consisting of: near infra red (NIR) reflectance, mid-infra red (IR) spectroscopy, light scattering, Raman scattering, polarimetry, photoacoustic spectroscopy, or other optical techniques. The sensing apparatus may also be based on a combination of several optical methods.

In one embodiment, the sensing apparatus includes an optical sensing apparatus, comprising a light-emitting unit, a measurement cell unit, a detector unit and a plurality of reflector units. The light-emitting unit may be provided with a source of light used for the optical measurement. The measurement cell unit contains the analyte-rich fluid, through which the light passes and in which the analyte concentration is measured. The measurement cell unit can be located either in that portion of the cannula that is located under patient's skin and is within the body or in that portion of the cannula that is located above the patient's skin and is outside the body.

The configuration in which the measurement cell resides within the body will be hereby referred-to as an “intrinsecus” configuration, and the configuration in which the cell resides outside the body, will be hereby referred-to as in “extrinsecus” configuration. The detector unit detects the light after it has passed through the measurement cell and is ready for analyte concentration analysis. The reflector units are used to direct the light along the optical path. Light originating from the light-emitting unit passes along an optical path through the analyte-rich fluid located in the measurement cell unit. This light returns to the detector unit, after passing through one or more reflectors.

In another embodiment, optical glucose monitoring is carried out in the “intrinsecus” configuration. Light passes from the light-emitting unit via analyte-rich fluid in the measurement cell unit, located in that portion of the cannula, which is located inside user's body.

In yet another embodiment, optical glucose monitoring is carried out in an “extrinsecus” configuration. The measurement cell unit is in that portion of the cannula, which is located above the skin. The optical path does not enter the body and the measurement cell is situated outside the body, above the skin.

In both “intrinsecus” and “extrinsecus” configurations, the light-emitting unit and the detector unit may be both located within the reusable part of the device.

### BRIEF DESCRIPTION OF THE DRAWINGS

FIG. 1A illustrates an optical absorption spectra of glucose in the NIR region for aqueous glucose after water subtraction, as shown in Journal of Biomedical Optics 5(1), 5-16 January 2000.

FIG. 1B illustrates an optical absorption spectra of glucose in the Mid-IR region for aqueous glucose after water subtraction, as shown in Journal of Biomedical Optics 5(1), 5-16 January 2000.

FIG. 1C illustrates the Raman spectrum for aqueous glucose after subtraction of the water background, as shown in Journal of Biomedical Optics 5(1), 5-16 January 2000.

FIG. 2 illustrates an exemplary non-invasive sensing device, coupled with a subcutaneous insulin delivery cannula, according to some embodiments of the present invention.

FIG. 3A illustrates an exemplary closed loop system, including the dispensing apparatus, the sensing apparatus, the processor-controller apparatus, and the remote control unit, with a single cannula, according to some embodiments of the present invention.

FIG. 3B illustrates an exemplary closed loop system, including the dispensing apparatus, the sensing apparatus, the processor-controller apparatus, and the remote control unit, in which the dispensing and sensing apparatuses have separate cannulae, according to some embodiments of the present invention.

FIG. 4 is an exemplary schematic view of a semi-permeable cannula and of the diffusion process, according to some embodiments of the present invention.

FIG. 5 is an exemplary schematic view of a permeable cannula and of the diffusion process, according to some embodiments of the present invention.

FIG. 6 is an exemplary schematic view of the cannula suitable for microdialysis or microperfusion, according to some embodiments of the present invention.

FIG. 7 illustrates an exemplary coaxial cannula, according to some embodiments of the present invention.

FIG. 8 illustrates an exemplary double lumen cannula, according to some embodiments of the present invention.

FIG. 9 illustrates an exemplary peristaltic pump with two tubes, corresponding to two separate cannulae—one for sensing the analyte and the other for dispensing fluid, according to some embodiments of the present invention.

FIGS. 10a-d illustrate exemplary insertion of the cannula into the body through a well arrangement, using a penetrating cartridge, according to some embodiments of the present invention.

FIGS. 11a-b illustrate an exemplary fluid delivery device having a reusable part and a disposable part, and optical sensing components deployed in these parts, according to some embodiments of the present invention.

FIGS. 12a-b illustrate two exemplary configurations of the location of the measurement cell—“intrinsecus” configuration and “extrinsecus” configuration, according to some embodiments of the present invention.

FIGS. 13a-b illustrate exemplary intrinsecus and extrinsecus configurations in a detailed view, as part of the whole system, according to some embodiments of the present invention.

FIG. 14 illustrates an exemplary “extrinsecus” configuration at the time when a measurement cell is deployed in the reusable part of the device, according to some embodiments of the present invention.

FIGS. 15a-b illustrate an exemplary device having one or more light sources and one or more detectors, according to some embodiments of the present invention.

FIGS. 16a-b illustrate exemplary light transfer from the light-emitting unit to the cannula through a lens, according to some embodiments of the present invention.

FIGS. 17a-b illustrate exemplary light transfer from the light-emitting unit to the cannula through two optical windows, according to some embodiments of the present invention.

FIG. 18 illustrates an exemplary MEMS spectrometer, according to some embodiments of the present invention.

FIGS. 19a-c illustrate an exemplary cannula provided with retro-reflectors plated by reflective coating, according to some embodiments of the present invention.

FIGS. 20a-b illustrate an exemplary cannula provided with retro-reflector configured as a tongue plated by reflective coating, according to some embodiments of the present invention.

FIGS. 21a-b illustrate an exemplary cannula with retro-reflector configured as prestressed flaps, according to some embodiments of the present invention.

FIG. 22 illustrates exemplary cladless optical fibers for transmitting light through the cannula, according to some embodiments of the present invention.

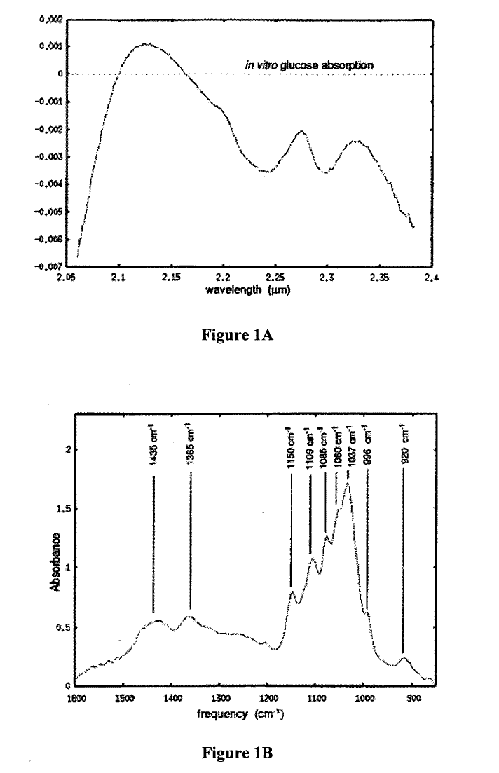

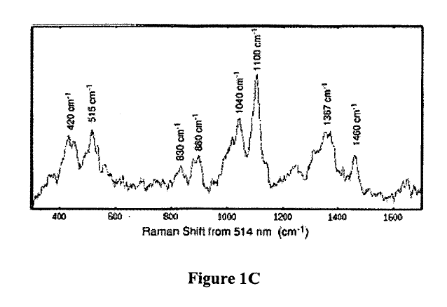

### DETAILED DESCRIPTION OF INVENTION

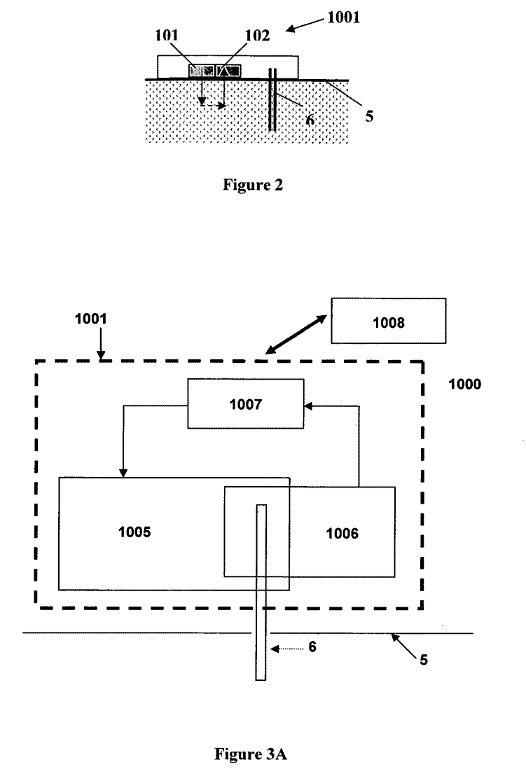

In some embodiments of the invention, the pumping apparatus is minimally-invasive and the sensing apparatus may be non-invasive. FIG. 2 illustrates a schematic drawing of a device (1001) adhered to the skin (5), according to some embodiments of the present invention. The device includes a cannula (6) for dispensing insulin and a non-invasive sensing apparatus. In some embodiments, the device (1001) includes all pumping and/or controlling elements (not shown in FIG. 2). The sensing apparatus includes a light-emitting unit (101), capable of illuminating light through a body tissue under the skin (5) and a detection unit (102), capable of detecting the returned light. The sensing apparatus monitors analyte (e.g., glucose) concentration levels and the sensed data is delivered to the processor-controller apparatus for pump programming and insulin delivery, for adjustment of analyte concentrations. As can be understood by one skilled in the art, insulin delivery means can be any subcutaneous delivery means such as needle micro-arrays, electrical stimulation, ultrasound and others.

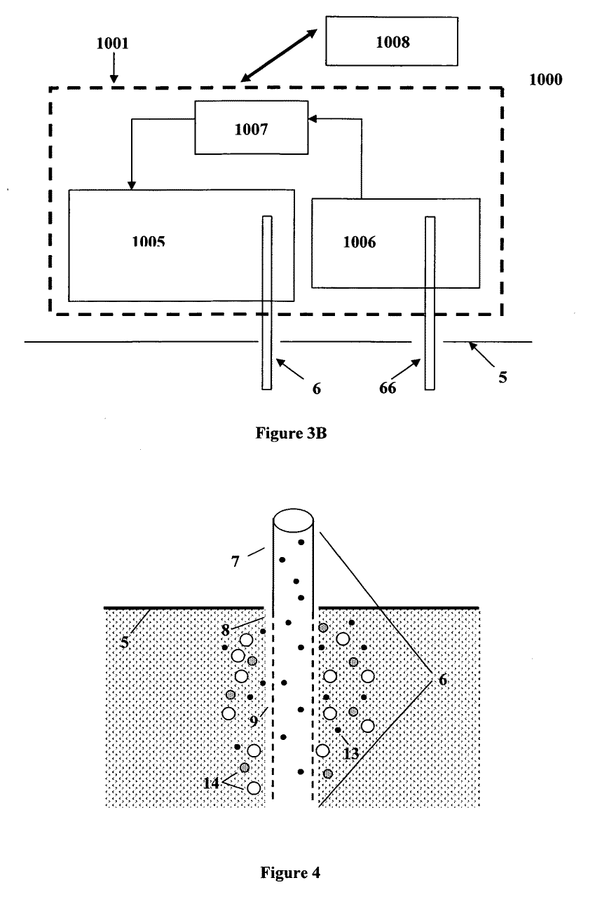

In some embodiments of the invention, the dispensing apparatus and sensing apparatus can be enclosed in a single device, and can use a single cannula to perform dispensing and sensing operations and can work as a closed-loop system. FIG. 3A illustrates various components of a closed loop or semi-closed loop system (1000) that has a dispensing apparatus (1005), a sensing apparatus (1006), a processor-controller apparatus (1007), a remote control unit (1008), and a cannula (6), where the cannula (6) (as shown in FIG. 3A) is located under the skin (5) in the subcutaneous tissue. In this embodiment, the system components, apart from the remote control unit (1008), can be configured to be enclosed within one device (1001), which can be adhered to the skin of the patient by adhesives (not shown in FIG. 3A). The remote control unit (1008) can be configured to maintain a bidirectional communication channel with the device (1001), thereby allowing programming, data handling, user/patient input, etc. A single cannula (6), which includes a permeable or semi-permeable membrane, is configured to penetrate the skin of the patient and allow concomitant fluid delivery to the body of the patient and sense analytes in the body of the patient. In a closed-loop system embodiment, the processor-controller apparatus (1007) is configured to receive input(s) from the sensing apparatus (1006) (i.e., analyte concentration) and, after processing the data, authorize the dispensing apparatus (1005) to dispense fluid accordingly. In a semi-closed-loop system embodiment, the processor-controller apparatus (1007) can be configured to receive input(s) from the patient, e.g., through the remote control unit (1008).

In other embodiments of the present invention, the device includes separate reusable and disposable parts (not shown in FIG. 3A), wherein each part can be enclosed in its own housing. In some embodiments, relatively cheap components of the sensing and dispensing apparatuses can be configured to be enclosed inside the disposable part and relatively expensive components of both apparatuses can be configured to be enclosed inside the reusable part.

In other embodiments, sensing of glucose levels and dispensing of insulin can be done through separate exit ports, using two cannulae that can be inserted into the subcutaneous tissue, residing in the body, as shown in FIG. 3B. The dispensing apparatus (1005) and sensing apparatus (1006) can be configured to have separate cannulae (6, 66). The dispensing apparatus (1005) can be configured to include some features of an insulin pump, such as a reservoir, a driving mechanism, tubing, etc., and a cannula (6). The sensing apparatus (1006) includes a reservoir containing isotonic fluid and a pump for dispensing the isotonic fluid through the permeable or semi-permeable cannula (66), allowing analyte concentration level measurements, as discussed above.

The processor-controller apparatus (1007) is configured to receive inputs from the sensing apparatus (1006) and from the patient/user (via the user control unit (1008) in the semi-closed loop configuration). The apparatus (1007) is further configured to control the dispensing apparatus (1005) to deliver insulin through its own cannula (6) to regulate glucose levels. In this embodiment, two cannulae (6, 66) are configured to be positioned next each other. The dispensing apparatus can deliver insulin by other means, in addition to or instead of a subcutaneous cannula, such as using a micro-array of miniature needles or any other trans-cutaneous delivery means such as electrical and ultrasound skin stimulation.

In some embodiments, the cannula that is used for sensing analyte concentration levels and for delivering fluid is semi-permeable. This means that the cannula allows diffusion of the analyte into the cannula. FIG. 4 schematically illustrates a structure of the cannula (6) having an upper portion (7) and a lower portion (8). The portions (7) and (8) can be configured to be disposed above and below the skin of the patient, respectively. FIG. 4 further illustrates diffusion of substances of various molecular weight under the skin of the patient. The lower cannula portion (8) can include a semi-permeable membrane (9) that is configured to allow substances with low molecular weight, e.g., a desired analyte (13), such as glucose, to pass through pores of the semi-permeable membrane (9). The membrane (9) can be configured to prevent substances (14) of higher molecular weight from passing through the pores.

The cannula (6) can be perfused with an analyte-free solution (e.g., insulin or saline) in order of diffusion to occur. In some embodiments, the diffusion of analyte molecules can occur across the semi-permeable membrane (9) because of an initial concentration gradient. As can be understood by one skilled in the art, the diffusion of molecules can occur due to other conditions and/or parameters. The diffusion process occurs in the direction of the concentration gradient until partial or full equilibrium between the inner and outer sides of the cannula is achieved. In some embodiments, the gradient is measured between the tissue fluid (e.g., ISF) and the solution within the cannula. The outcome of the diffusion process is the presence of solution enriched by the analyte (i.e., the dialysate), inside the cannula (6) with an analyte concentration. The analyte concentration can be proportional or equal to the analyte concentration in the ISF. The analyte (e.g., glucose) concentration levels can be optically measured either immediately in the portion of the cannula that is inside the body (i.e., “intrinsecus” configuration). Alternatively, the concentration levels can be measured by transporting the fluid above the skin and measuring the glucose concentration in a location outside the body (i.e., “extrinsecus” configuration).

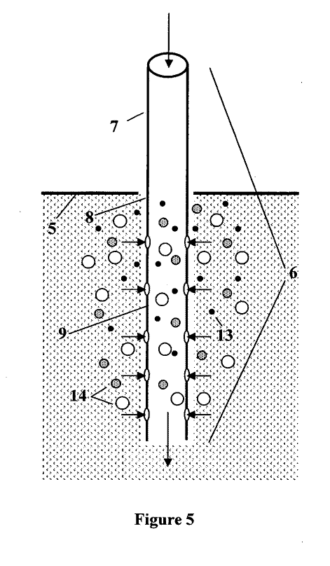

In other embodiments, the cannula that is used for sensing analyte concentration levels and for delivering fluid is permeable. This means that in addition to the diffusion of analyte molecules from the ISF into the cannula (13) (e.g., glucose), additional analytes contained in the ISF (14) can also diffuse into the cannula (13). FIG. 5 schematically illustrates exemplary structure of the cannula (6) having upper (7) and lower (8) portions that are disposed above and below the skin, respectively, as well as a diffusion of molecules having variable weight. The lower cannula portion (8) can include a permeable membrane (9) allowing the ISF to pass through pores of the permeable membrane (9). The cannula (6) can be perfused with a solution (e.g., insulin or saline). Diffusion of ISF occurs across the permeable membrane (9) because of an initial concentration gradient or any other reasons. The diffusion process occurs in the direction of the concentration gradient, between the tissue fluid (e.g., ISF) and the solution within the cannula, reaching partial or full equilibrium between the inner and outer sides of the cannula. The outcome of the diffusion process is the presence of a solution enriched by the analyte (i.e., the dialysate), inside the cannula (6) with an analyte concentration, which is proportional or equal to the analyte concentration in the ISF. One of the advantages of such cannula is that it is provided with larger pores that enable cheaper and easier manufacturing of the cannula.

In other embodiments, the cannula that is used for sensing analyte concentration levels and for delivering fluid can be a microdialysis or a microperfusion probe. The probe can be perfused with a solution (e.g., insulin or saline). The outer membrane of the probe may be either semi-permeable or permeable.

FIG. 6a illustrates a microperfusion probe having a semi-permeable membrane. FIG. 6b illustrates a microperfusion probe having a permeable membrane.

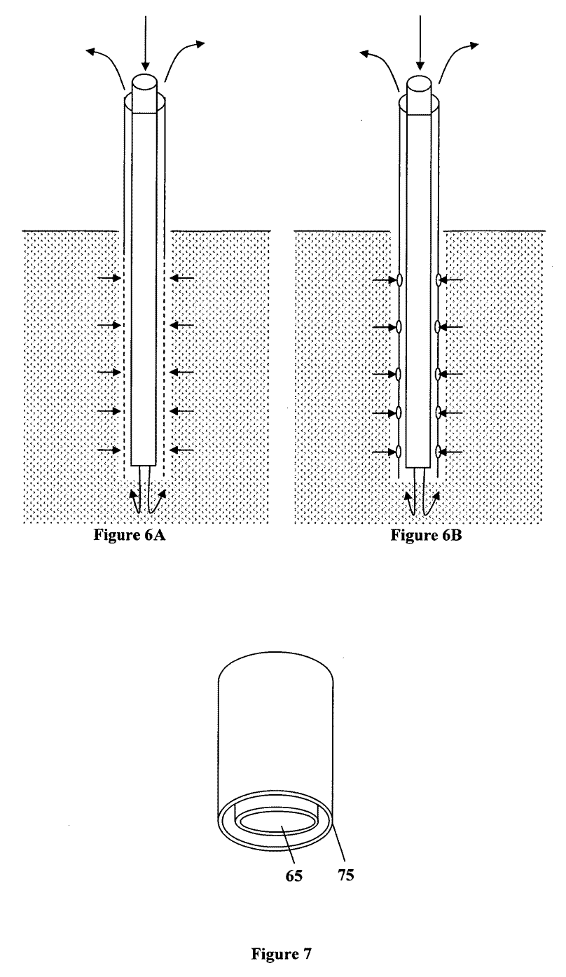

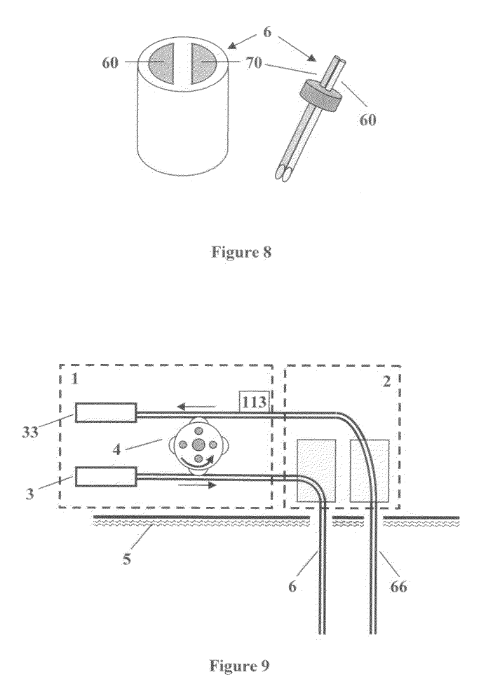

In another embodiment of the present invention, the cannula that is used for sensing analyte concentration levels and for delivering fluid is coaxial. The cannula can be provided with an inner part (65) surrounded by an outer part (75), as shown in FIG. 7. The inner part (65) of the cannula (6) is used to deliver fluid (e.g., insulin) and the outer part (75) is used to sense analyte levels (e.g., glucose). In this embodiment, the outer part of the cannula may be permeable or semi-permeable. Alternatively, the inner part (65) can be used to sense analyte levels (e.g., glucose) and the outer part (75) can be used to deliver fluid (e.g., insulin).

In some embodiments, the sensing of analyte (e.g., glucose) levels and the dispensing of fluid (e.g., insulin) can be both carried out by a single double lumen cannula, containing two compartments that are separated by a partition. This double lumen cannula includes one compartment dedicated to sensing (60) and another compartment dedicated to dispensing (70). FIG. 8 is a schematic drawing of an exemplary double-lumen cannula (6) with one compartment dedicated to sensing glucose (60) and the other compartment dedicated to dispensing insulin (70), according to some embodiments of the present invention.

In some embodiments, the dispensing apparatus and sensing apparatus each include independent cannulae (6, 66) and respective associated reservoirs (3, 33). The cannulae (6, 66) can be configured to share a common peristaltic pump (4). The pump (4) can be configured to displace fluid in more than one tube, in a space-saving configuration, as shown in FIG. 9. One tube can be part of the sensing apparatus and can be further used to deliver fluid from the sensing cannula (66) to the spectrometer (113), and then to the collecting reservoir (33). The other tube can be part of the dispensing apparatus and can be further used to deliver fluid from the delivery fluid reservoir (e.g., insulin reservoir) (3) to the body via the delivery cannula (6). The dispensed and sensed fluids can be configured to remain inside the tubing at all times. This feature prevents mixing of the fluids pumped from different reservoirs, thus, sufficiently reducing the risk of contamination, permitting control over the content and purity of the fluid delivered to the patient. In other embodiments, the collecting reservoir and the delivery fluid reservoir can be combined into a single reservoir.

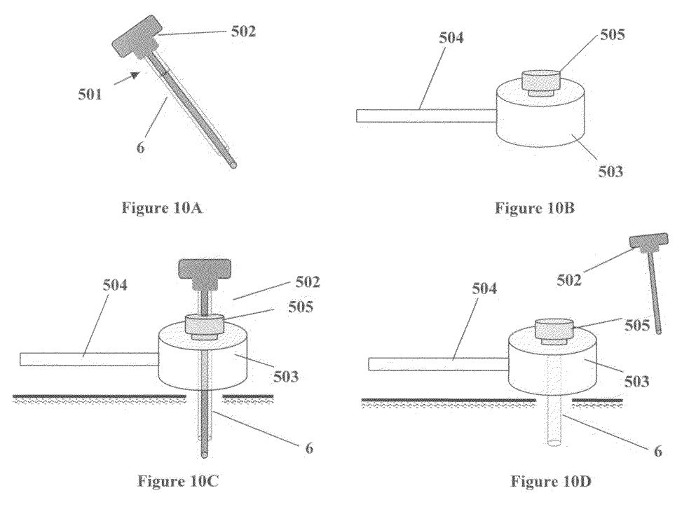

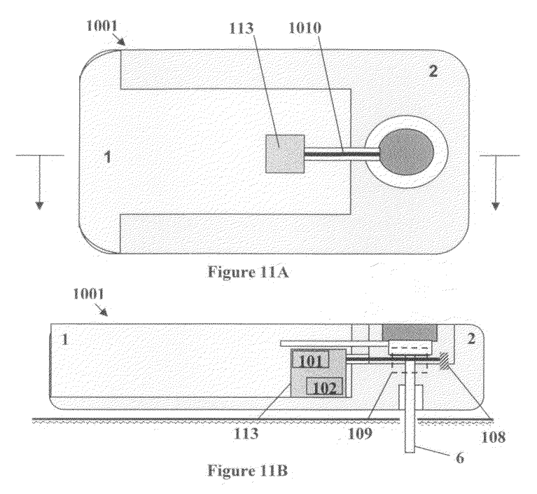

In some embodiment, the fluid delivery device can be inserted into the body using a penetrating cartridge (501), which contains a penetrating member (502) and a cannula (6), as shown in FIGS. 10a-d. In alternate embodiments, a “well” arrangement (503) can be used to provide fluid communication between the delivery tube (504) and the cannula (6) which resides in the subcutaneous tissue. The “well” arrangement (503) has an opening on the top, which is closed by a sealing plug (505). When the penetrating cartridge (501) is inserted into the well arrangement (503), it pierces the sealing plug (505). The well arrangement (503) also has an inlet port on its side and a channel, allowing the passage of fluid from the tube (504) to the cannula (6), though a lateral opening made in the cannula.

An explanation of the well-arrangement and the penetrating cartridge can be found in co-pending and co-owned U.S. patent application Ser. No. 11/397,115, the disclosure of which is incorporated herein by reference in its entirety.

FIG. 10a illustrates an exemplary penetrating cartridge (501) prior to insertion, including the penetrating member (502), and the cannula (6). FIG. 10b illustrates an exemplary well arrangement (503) prior to insertion, the rubber plug (505), and the delivery tube (504). FIGS. 10c illustrates an exemplary penetrating cartridge (501) and “well” arrangement (503), during penetration into the skin (5). FIG. 10d illustrates an exemplary cannula (6) being inserted into the skin (5) and connected to the well arrangement (503), and then plugged by the rubber plug (505) and connected to the delivery tube (504) (after removal of the penetrating member (502)).

In yet other embodiments, the device (1001) includes two parts—a reusable part (1) and a disposable part (2), as shown in FIGS. 11a-b. The reusable and/or disposable parts can be configured to include an optical sensing apparatus that can further include a plurality of units. In some embodiments, relatively expensive, non-disposable elements of the optical sensing apparatus, can be configured to be disposed within the reusable part (1) of the device. FIG. 11a is a top view of an exemplary device with the reusable (1) and the disposable (2) parts, according to some embodiments of the present invention. FIG. 11b is a side view of such device. As can be understood by one skilled in the art, the illustration of the two-part device (1001) is for exemplary, non-limiting purposes, and as such, the present invention can include more than two parts for the device (1001).

As illustrated in FIGS. 11a-b, the spectrometer (113) includes a light-emitting unit (101) that can serve as a light source for the optical measurement, and a detector unit (102) that can detect a returned light after it passed through the analyte-rich fluid. The detector unit (102) can be configured to analyzes the returned light. Both the light-emitting unit (101) and the detector unit (102) can be disposed inside the reusable part (1) of the device (1001). The measurement cell unit (109) includes the analyte-rich fluid. The light can be directed through the analyte-rich fluid for performing an optical measurement. Reflectors (108) can be configured to direct the light between the different units of the optical sensing apparatus. These reflectors reside either in the reusable part (1) or the disposable part (2).

In some embodiments, light originating from the light-emitting unit (101) in the reusable part (1) can pass through the fluid located in the measurement cell unit (109) to reflector units (108). The reflector units (108) further direct the light through an optimized optical path (1010) to the detector unit (102). The detector unit (102) then analyzes the produced light spectra.

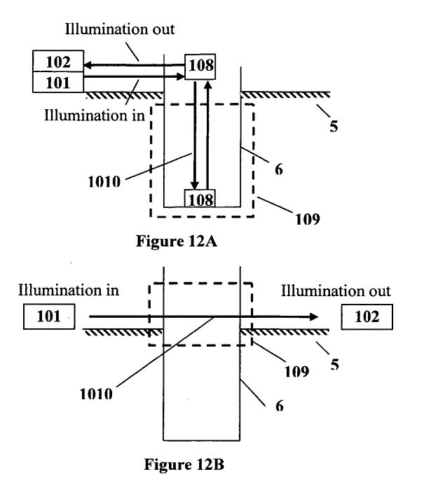

The optical sensing apparatus can be configured to measure analyte concentration using the emitted light. FIGS. 12a-b are schematic views of the two exemplary configurations of the location of the measurement cell (109)—“intrinsecus” and “extrinsecus”, respectively. A measurement cell in an “intrinsecus” configuration resides in a portion of the cannula (6) that is inside the body of the patient and under the surface of the skin (5), as illustrated in FIG. 12a. A measurement cell in an “extrinsecus” configuration resides in a portion of the cannula (6) that is located outside the body of the patient and above the surface of the skin (5), as illustrated in FIG. 12b.

FIGS. 13a-b are more detailed views of exemplary “intrinsecus” and “extrinsecus” configurations of the measurement cell, according to some embodiments of the present invention. FIG. 13a illustrates an exemplary “intrinsecus” configuration, in which the emitted light is transferred from the light-emitting unit (101), located in the reusable part (1), to the measurement cell unit (109), located inside the cannula (6), via an optical path (1010). The measuring cell is located in a portion of the cannula that is under the skin. The cannula is in fluid communication with the delivery reservoir (not shown in FIGS. 13a-b) via a “well” arrangement (503). The cannula (6), the well arrangement (503), the measurement cell (109), and a portion of the retro-reflectors (108) can be configured to be located within the disposable part (2) of the present invention's device. The light source (101), detector (102) and a portion of the optical path (1010) can be configured to be located in the reusable part (1) of the present invention's device. Reflector units (108) can be used in creating optical path (1010) between the light source (101), the sample in the measurement cell (109) and the detector (102). FIG. 13b illustrates an exemplary illumination of light through the measurement cell (109), located in the part of the cannula (6) that resides outside the body, in an “extrinsecus” configuration. In this configuration, the emitted light is transferred from the light-emitting unit (101), through the measurement cell unit (109), and to the detector (102), via the optical path (1010). In this configuration all of these components are located inside the present invention's device and above the skin.

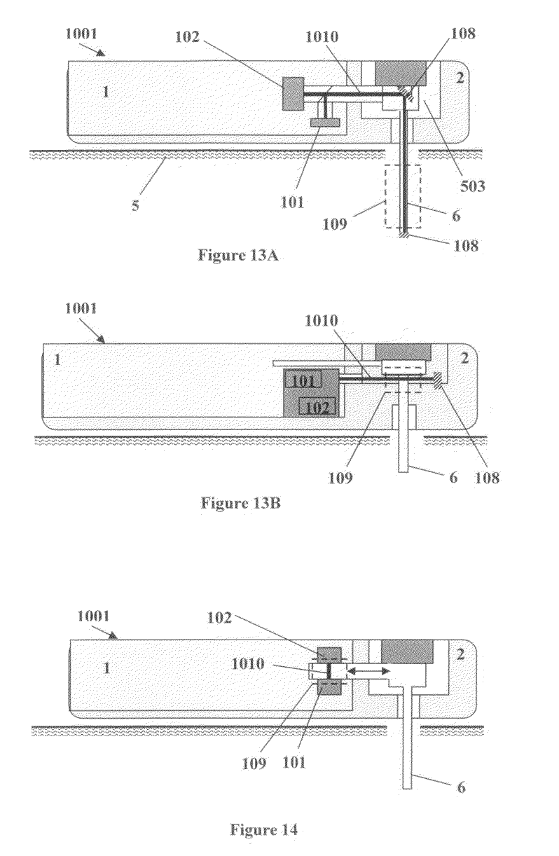

FIG. 14 illustrates an exemplary “extrinsecus” configuration in which the analyte-rich fluid is transported from the cannula to a measurement cell residing between the light-emitting unit (101) and the detector unit (102). In this configuration, the units (101) and (102) can be configured to be disposed inside the reusable part (1) of the device (1001). In this embodiment, the measurement cell unit (109) is located within the reusable part (1) of the device (1001).

In an “extrinsecus” configuration embodiment, the analyte-rich solution, residing inside the cannula after diffusion, is transported to the upper portion of the cannula, to be analyzed in a measurement cell located above the skin. For transporting the analyte-rich fluid up the cannula to the measurement cell, in the “extrinsecus” configuration, the pump within the device is used for pumping the fluid up and down the cannula.

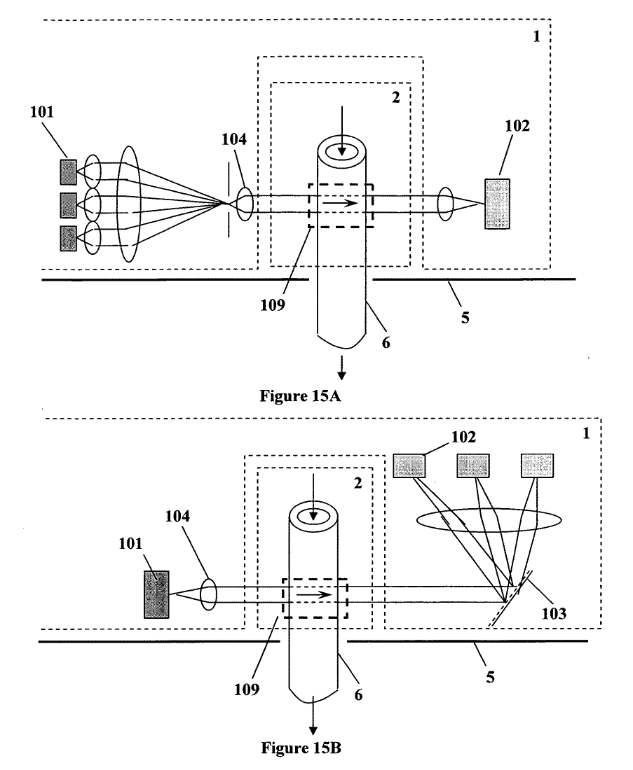

In another embodiment of the present invention, the optical sensing technique involves a use of one or more light-emitting sources, which produce illuminating light to be detected by one or more detectors, as shown in FIGS. 15a-b. FIG. 15a illustrates an exemplary device that includes a plurality of light-emitting sources (101), which emit light that can be detected using a single detector (102). In this embodiment, light is transmitted from the light-emitting unit (101), located in the reusable part (1) of the device, through an optical fiber or via a mirror (104), to the measurement cell (109) within the cannula (6), located in the disposable part (2) of the device. The transmitted light passes through the analyte-rich solution, residing in the measurement cell (109), to the detector unit (102), located in the reusable part (1) of the device. Prior to reaching the detector unit (102), the light can be directed through a grating that separates the light into its spectral components. The spectral components can be detected using matching detectors.

FIG. 15b illustrates an exemplary device that includes a single light-emitting source (101) and a plurality of detectors (102), according to some embodiments of the present invention. Each source (101) can be configured to emit radiation at a discrete wavelength (or a narrow range of wavelengths). The emitted light is transmitted from the light-emitting unit (101) through an optical fiber or via mirrors (104) to the measurement cell (109). The light passes through the measurement cell (109), and through the analyte-rich solution residing in it, and is detected by detectors (102).

Examples of detectors include Silicon, InGaAs, PbS, PbSe and bolometric detectors, or any other detectors. As can be understood by one skilled in the art, any detector operating in the desired spectral range can be incorporated in the present invention's device. For example, some bolometric detectors are manufactured by SCD Ltd., Israel. Gratings are available from Edmund Optics, USA.

Examples of light emitting sources include white LEDs, semiconductor lasers having a specific spectral range and VESCLs, or any other light emitting sources. In addition, organic light sources, such as OLED and electrofluorescence material, can be incorporated into the device. Light sources are available from OSRAM Germany, NICHIA Japan and others.

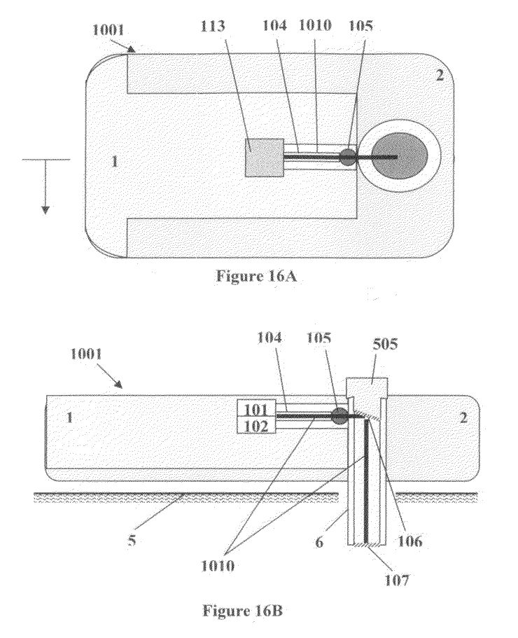

In some embodiments, the light is transported from the light emitting unit (101) in the reusable part (1), through the measurement cell residing inside the cannula (6) in the disposable part (2) and back to the detector unit (102), residing in the reusable part (1), using two units of reflectors (106, 107). The reflectors (106, 107) can be configured to be proximal and distal, respectively, to the surface of the skin (5), as shown in FIGS. 16a-b. FIG. 16a-b further illustrate a side-view and a top-view of the lens configuration, respectively.

The proximal reflector (106) can be affixed to the sealing plug (505) that seals the well arrangement (503). The distal reflector (107) can be deployed within the cannula (6) at its bottom and away from the surface of the skin. To receive maximum reflection possible, retro-reflectors can be used.

The light can be transmitted from the light-emitting unit (101) to the proximal reflector (106) through an optical fiber (104) terminating at a lens (105). The lens (105) is located at the connection region between the reusable part (1) and the disposable part (2). The lens is positioned at the lateral side of the reusable part (1), being adjacent to the cannula (6), which is located in the disposable part (2). The proximal reflector (106) directs the light into the cannula (6), such that it passes through the analyte-rich fluid, and hits the bottom of the cannula (6), where the distal reflector (107) is located. The distal reflector (107) directs the light back through the cannula (6) to the proximal reflector (106). The latter directs the light through the lens (105) and optical fiber (104) to the detector unit (102) in the reusable part (2).

In some embodiments, the lens (105) can be configured to have no optical force, i.e., no ability to scatter or focus light. In other embodiments, the lens can be configured to have the ability to focus light.

The lens can be made from an IR transmitting plastic, glass or crystal. Use of plastic lens can be more attractive because of its low cost, however, glass and crystal lens have superior optical properties. As can be understood by one skilled in the art, other materials can be used.

The walls of the cannula (6) can be made of a material that does not absorb the light with wavelengths corresponding to the light emitted from the light-emitting unit (101). This allows the light to pass into the cannula (6).

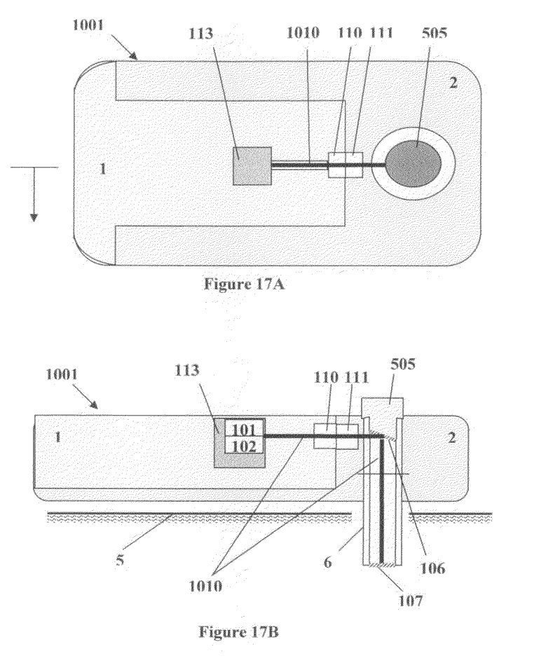

In yet another embodiment of the device (1001), the components of the sensing apparatus can be deployed in the reusable and disposable parts, as illustrated in FIGS. 17a-b. In this embodiment, two optical windows (110, 111) are disposed inside the reusable part (1) and disposable part (2), respectively. The windows are configured to transmit light from the light emitting unit (101), located in the reusable part (1), to the disposable part (2), as shown in FIGS. 17a-b. The light-emitting unit (101), the detector unit (102) and other components responsible for optical detection, can be configured to be disposed inside the spectrometer (113), located in the reusable part (1).

The optical windows (110, 111) can be manufactured from a material that does not absorb wavelengths corresponding to the light emitted from the light-emitting unit (101), thus, allowing the light to pass through them. The optical windows can be located at the connection region between reusable (1) and disposable (2) parts and can be exactly aligned with each other, as shown in FIGS. 17a-b. This allows passage of the light from the reusable part (1) to the disposable part (2) and back. For illustrative purposes, the window (110), located in the reusable part (1), can be referred to as an R-window (110) (“R” stands for reusable) and the window (111), located in the disposable part (2), can be referred to as a D-window (111) (“D” stands for disposable). The optical windows (110, 111) can serve as focusing means, for narrowing down the scattering of the emitted and returning light.

Optical windows (110, 111) can be manufactured from IR transmitting plastic, glass or crystal, or any other suitable material. Plastic is advantageous to use due to its low cost, yet glass and crystals have superior optical properties. As can be understood by one skilled in the art, optical windows can be manufactured from other suitable materials.

As shown in FIGS. 17a-b, the light travels in an optical path (1010) from the light-emitting unit (101) to the R-window (110) and through the D-window (111). After passing through both optical windows, the light encounters the proximal reflector unit (106), which directs the light into the cannula (6) and through the measurement cell containing the analyte-rich fluid residing inside the cannula (6). The distal reflector unit (107), provided at the bottom of the cannula (6), reflects the light back to the skin (5) surface. The proximal reflector unit (106) directs the light back through the windows (110, 111) and to the detector (102). For receiving maximum reflection possible, retro-reflectors can be used.

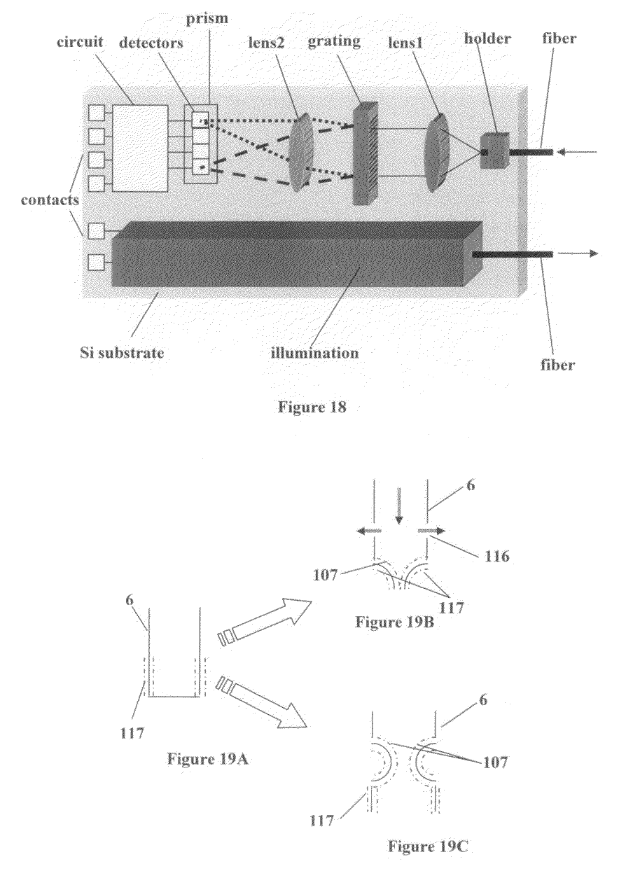

In an embodiment, the spectrometer (113) can be a MEMS spectrometer, containing appropriate micro-electro-mechanical components that produce illuminating light, detect reflected light, as well as lenses and gratings, as shown in FIG. 18. As illustrated in FIG. 18, the light passes through a holder onto lens1, which reflects it towards a grating. Then, the grated light is then reflected towards lens2. The light is then reflected towards a prism having a plurality of detectors coupled to a circuit configured to analyze the reflected light. The initial illumination can be produce using an silicon substrate.

In one embodiment, the cannula may be provided with a retro-reflection capability. This can be achieved by coating the cannula interior with a reflective plating, which serves as a reflector.

FIGS. 19a-c illustrate an exemplary distal reflector (107), which can be arranged by coating the bottom part of the cannula with gold plating. The plating can serve as a reflector (107), which reflects the light inside the cannula (6) and, thus, a retro-reflection effect is achieved.

In an embodiment of FIG. 19a, only the bottom portion of the cannula (6) is plated (117), rather than the entire cannula. In some embodiments, the cannula (6) can be narrowed at the bottom, so that the plated retro-reflector (117) resides at the bottom of the cannula (6), as shown in FIG. 19b. Lateral openings (116) can be made on the side walls of the cannula (6) to permit outflow of the fluid through these openings (116).

In the embodiment of FIG. 19c, the cannula is narrowed on its side walls, so that the reflector (117) is constituted by the narrowed sides of the cannula (6). The outflow of the fluid is possible through the open bottom of the cannula (6). As can be understood by one skilled in the art, FIGS. 19a-c illustrate exemplary non-limiting embodiments of cannula (6), and other configurations of cannula (6) are possible.

In an embodiment, the retro-reflection of light from the bottom of the cannula (6) is achieved by virtue of a reflective elastic tongue (115) attached at the bottom part of the cannula, as illustrated in FIGS. 20a-b. The reflective tongue (115) can be a leaf spring. During insertion and withdrawal of the penetrating member (502), the tongue (115) can be directed perpendicular to the lateral walls of the cannula (6). The reflective tongue (115) serves as the distal reflector (107). FIG. 20a is a bottom view of the cannula. FIG. 20b is a side view of the cannula and the tongue.

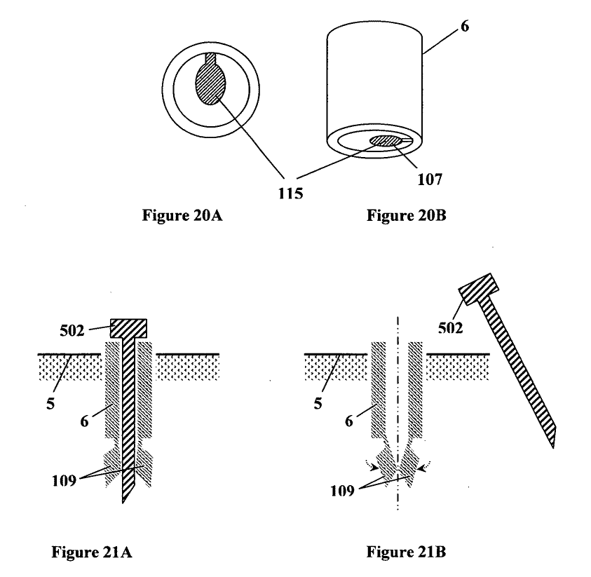

In an alternate embodiment, retro-reflection of light from the bottom of the cannula (6) is achieved by virtue of elastically foldeable pre-stressed flaps (109) provided at the bottom of the cannula, as illustrated in FIGS. 21a-b. The flaps (109) are coated by a reflective material, on their inner side, making them retro-reflective.

In their pre-stressed position, the flaps remain together, at an angle suitable for reflection of light upwards. As shown in FIG. 21a, prior to insertion of the cannula (6) using the penetrating member (502) to the body, through the skin (5), the flaps are separated by the penetrating member (502). Upon its withdrawal, the flaps (109) on the cannula (6) are elastically folded to their pre-stressed position, as shown in FIG. 21b, and serve as retro-reflectors to incoming light.

In yet another alternate embodiment, the light is transmitted into the cannula (6) and from the cannula (6) by virtue of one or more optical fibers (300) that are inserted in the lateral walls of the cannula (6) and extend therealong. The clad is removed from these optical fibers (300) at several locations along the fiber where the cannula (6) is under the skin (5), thus providing an array of clad-less fibers.

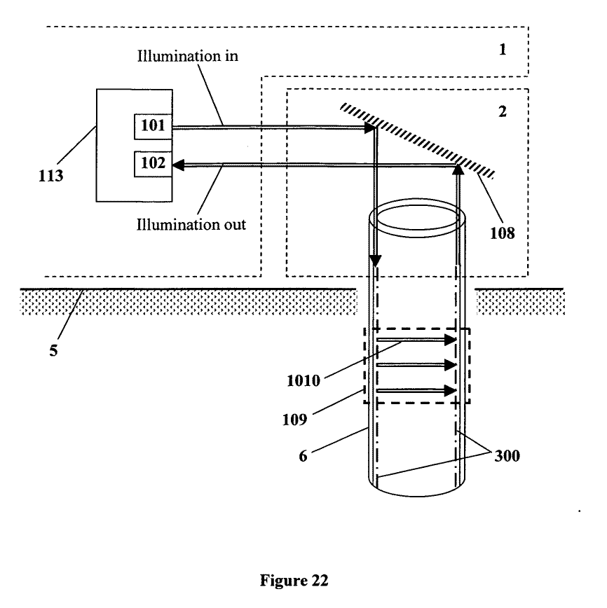

FIG. 22 illustrates the above embodiment in further detail. The light leaves the light-emitting unit (101) in the spectrometer (113), located in the reusable part (1) of the device, passes to the reflector unit (108) in the disposable part (2) of the device, and into the optical fibers (300) in the cannula (6). In that part of the cannula (6), which is inside the body, the light leaves the optical fibers (300) from the clad-less locations on the fibers, and enters the measurement cell (109), transmitted through the analyte-rich fluid. The light is collected by the clad-less fibers (300) on the opposite side of the cannula (6), travels up the optical fibers (300) and makes its way back through the reflector (108) in the disposable part (2), to the detector (102) in the reusable part (1). In this embodiment, the length of the optical path (1010) is defined by the cannula diameter, i.e., by the distance between opposite clad-less locations provided at optical fibers (300).

The fibers (300) are clad-less partially, thus, light diffuses out of the illuminating fiber and, after passing through the glucose carrying fluid in the measurement cell (109), and getting imprinted by the glucose, is partially captured by the receiver fiber, for the purpose of sensing.

Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed:

1. A system for continuous monitoring of one or more body analytes and controlling delivery of fluids to a body of a patient, comprising:
a sensing apparatus configured to detect concentration level of an analyte in the body of the patient using optical means; and
a dispensing apparatus configured to infuse fluid into the body of the patient based on the detected concentration level of the analyte, wherein
the sensing apparatus and the dispensing apparatus use a subcutaneous cannula.

2. The system according to claim 1, further comprising a controller configured to regulate dispensing of a fluid based on a detected concentration level of analyte in the body of the patient.

3. The system according to claim 2, wherein the controller is further configured to regulate dispensing of a fluid based on a detected concentration level of analyte in the body of the patient and external inputs.

4. The system according to claim 1, wherein the optical means comprises spectroscopic analysis means.

5. The system according to claim 1, wherein the analyte is glucose.

6. The system according to claim 1, wherein the infused fluid is insulin.

7. The system according to claim 1, wherein the sensing apparatus is a non-invasive sensing apparatus configured to non-invasively detect concentration level of analyte.

8. The system according to claim 1, wherein the sensing apparatus is a minimally-invasive sensing apparatus configured to detect concentration level of analyte, and the sensing apparatus includes a cannula configured for insertion into a subcutaneous tissue of the skin of the patient and to monitor level of interstitial fluid (“ISF”) analyte.

9. The system according to claim 8, wherein the sensing apparatus is a skin adhesive device is configured to utilize micropores made in the skin of the patient to monitor level of ISF analyte.

10. The system according to claim 8, wherein the cannula comprises a semi-permeable membrane configured to enable diffusion and selectively allow entry of analyte molecules into the cannula, wherein a concentration of analyte in the fluid within the cannula is substantially proportional to a concentration of analyte in the interstitial fluid outside the cannula.

11. The system according to claim 1, wherein the optical means is configured to spectroscopically analyze the analyte using technology selected from the group consisting of: near infra red (NIR) reflectance, mid-infra red (IR) spectroscopy, light scattering, Raman scattering, polarimetry, and photoacoustic spectroscopy.

12. The system according to claim 11, wherein the optical means further comprises:
at least one light-emitting unit having a source of light;
a measurement cell unit for receiving an analyte-rich fluid, said measurement cell unit is configured to allow passage of light for measurement of level of concentration of analyte;
at least one detector unit configured to detect the light after it passed through the measurement cell unit prior to analysis of level of concentration of analyte; and
at least one reflector unit configured to direct light emitted by the light-emitting unit through the analyte-rich fluid located in the measurement cell unit.

13. The system according to claim 12, wherein the measurement cell unit is configured to be placed in that portion of the cannula, which is inside the body of the patient.

14. The system according to claim 12, wherein the measurement cell unit is configured to be placed in that portion of the cannula, which is outside the body of the patient.

15. A method for continuous monitoring of one or more body analytes and controlling delivery of fluids to a body of a patient, comprising:
detecting concentration level of an analyte in the body of the patient; and
infusing fluid into the body of the patient based on the detected concentration level of the analyte;
performing said detecting and said infusing a subcutaneous cannula.

16. The method according to claim 15, further comprising
regulating dispensing of a fluid based on a detected concentration level of analyte in the body of the patient.

17. The method according to claim 15, wherein the analyte is glucose.

18. The method according to claim 15, wherein the infused fluid is insulin.

19. The method according to claim 15, wherein said detecting further comprises
non-invasively detecting concentration level of analyte.

20. The method according to claim 15, wherein said detecting further comprises
minimally-invasively detecting concentration level of analyte.

21. The method according to claim 20, further comprising
inserting a cannula into a subcutaneous tissue of the skin of the patient; and
monitoring level of an interstitial fluid (“ISF”) analyte.

22. The method according to claim 21, wherein said detecting further comprises
utilizing micropores made in the skin of the patient to perform said monitoring of level of ISF analyte.

23. The method according to claim 22, wherein the cannula includes a semi-permeable membrane configured to enable diffusion and selectively allow entry of analyte molecules into the cannula, wherein a concentration of analyte in the fluid within the cannula is substantially proportional to a concentration of analyte in the interstitial fluid outside the cannula.

24. The method according to claim 15, wherein said detecting further comprises
spectroscopically analyzing the analyte using technology selected from the group consisting of: near infra red (NIR) reflectance, mid-infra red (IR) spectroscopy, light scattering, Raman scattering, polarimetry, and photoacoustic spectroscopy.

25. The method according to claim 24, wherein said spectroscopically analyzing further comprises
emitting light using a light-emitting unit having a source of light;
using a measurement cell unit for receiving an analyte-rich fluid, allowing passage of light for measurement of level of concentration of analyte;
using a detector unit, detecting the light after the light passed through the measurement cell unit prior to analysis of level of concentration of analyte; and
using a reflector unit, directing light emitted by the light-emitting unit through the analyte-rich fluid located in the measurement cell unit.

26. The method according to claim 25, wherein the measurement cell unit is configured to be placed in that portion of the cannula, which is inside the body of the patient.

27. The method according to claim 25, wherein the measurement cell unit is configured to be placed in that portion of the cannula, which is outside the body of the patient.
